(19) United States
(12) Patent Application Publication
(10) Pub. No.: US 2008/0031967 A1
(43) Pub. Date: Feb. 7, 2008

(54) Compositions, products and methods for controlling weight in a mammal

(76) Inventors: Charles Carson Bascom, Hamilton, OH (US); Robert Scott Youngquist, Mason, OH (US); Amy Ann Walanski, Fairfield, OH (US); Begonia Y. Ho, Cincinnati, OH (US); Gary Richard Fuentes, Batesville, IN (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/973,423

(22) Filed: Oct. 9, 2007

Related U.S. Application Data
(63) Continuation of application No. 11/032,341, filed on Jan. 10, 2005.

(51) Int. Cl. A61K 35/60; A61K 31/19; A61K 31/215; A61K 31/352; A61K 31/40; A61P 43/00; A61K 36/82; A61K 31/426; A61K 31/44; A61K 31/52

(52) U.S. Cl. 424/523; 424/729; 514/263.1; 514/334; 514/370; 514/408; 514/424; 514/456; 514/468; 514/552; 514/557

(57) ABSTRACT

Compositions for controlling mammalian weight, said compositions comprising one or more lipolytic agents for stimulating mammalian lipolysis and/or thermogenic agents for modulating mammalian thermogenesis. Products, consumer and otherwise, comprising said compositions and methods of using both the present compositions and products to control mammalian weight.

### CROSS REFERENCE TO RELATED APPLICATION

This application is a continuation of U.S. application Ser. No. 11/032,341, filed Jan. 10, 2005, now pending.

### FIELD OF THE INVENTION

The present invention relates to compositions, products and methods for controlling weight in a mammal. Specifically, the present invention relates to compositions comprising one or more agents for stimulating mammalian lipolysis and/or modulating mammalian thermogenesis. The present invention further relates to products, consumer and otherwise, comprising said lipolytic and/or thermogenic agents, as well as methods of using both the compositions and products disclosed herein. The compositions and products of the present invention are adapted to encourage localized weight loss, body sculpting, and/or anti-cellulite benefits in the mammalian subjects to which they are applied.

### BACKGROUND OF THE INVENTION

With approximately 61% of adults overweight and 28% obese, and with 13% of kids age 6 to 8 and 14% of adolescents overweight, consumers continue to seek new weight control products that are convenient and that provide an effective solution to weight loss. The weight loss market now offers a multitude of new products, food-based and otherwise, to address consumers' changing motivations, practices, and demands. While historically the weight loss market has cycled—allowing certain segments of the market to achieve greater success than others—recent market data now suggests that sales in all segments of the weight loss industry are on the rise. Over the past few years, the following segments of the weight loss industry experienced significant growth: diet soft drinks, artificial sweeteners, health club memberships, weight-loss centers, medically-supervised diet programs, anti-obesity prescription drugs, low-calorie diets, low-calorie entrees, retail and multi-level meal replacements and appetite suppressants, and diet books, cassettes and exercise videos.

The majority of sales in the weight loss market in the recent past are attributed to ‘lesser evil’ foods, such as low fat, low calorie, sugar free, diet sodas. Weight loss supplements, primarily offered in pill and liquid meal replacement form, have also experienced significant growth in the past few years. Indeed, liquid meal replacement supplements currently account for approximately half of all weight-loss supplement sales. Such supplements are shelf stable liquid nutritional formulas created primarily to serve as meal substitutes. Another segment that experienced surprising growth in recent years is the supplement pill segment. This category encompasses diet pills and herbal formulations with ma huang (i.e., ephedra). It is believed that such pills and formulations accelerate calorie consumption and suppress the appetite. Currently, most weight-loss pill-form supplement are made up of herbal blends or mixed formulas. Such blends typically contain one or more of the following ingredients: chromium, chromium picolinate, CLA, HCA, guarana, caffeine, ginseng, green tea, chitosan, cholecystokinin/CCK, synephrine and ephedra. They may also contain vitamins, minerals, fibers, inositol, chitin, sports nutrition ingredients, psyllium, aloe, senna, willobark, Garcinia cambogia, yerba mate, Sida condifolia, kola nut, Bladder wrack, yohimbe, Commiphora mukal, Piper Longum, burdock, dandelion, nettles and others. Nevertheless, many diet pills and herbal formulations, particularly those containing ephedra, have been linked with adverse health effects, while others have simply proven to be ineffective in controlling mammalian weight.

Market data further suggests that consumers have attempted to control weight loss via careful selection of household groceries. Indeed, many consumers have increased their consumption of low calorie foods, including functional foods and nutraceuticals. Other consumers have relied upon the use of topical and/or localized weight loss products, including body-sculpting creams and the like. This segment of the weight control market has experienced stagnant growth, due largely in part to the minimal efficacy levels conventionally associated with such products. Other weight loss trends have emerged in recent years—including the high protein, low carbohydrate weight loss regimes led by the Atkins Diet and others. Moreover, many high satiety food products have been launched to address consumers' need to stay “full” longer and avoid unhealthy snacking. Such food products typically contain so-called calorie reduction agents, consisting of fat reducers and non-nutritive sweeteners. There are few meaningful drug and/or clinical-based options for managing weight and obesity on the market today. Contemporary drug-based therapies for weight loss are focused on appetite suppression and/or inhibition of fat adsorption. Drug-based therapies have been associated only with moderate efficacy, while the high cost of clinical-based options makes them unavailable to most consumers.

Thus, although an abundance of weight control products are available on the market, only a few have proven to be actually effective in controlling mammalian weight. Moreover, contemporary supplement pills, drugs and topical weight control products are often associated with adverse health risks and little clinical data demonstrating their efficacy. Accordingly, there remains a significant and unmet need in the art to identify and deploy efficacious compositions and products for controlling mammalian weight. Such compositions and products should provide a wide array of weight control benefits to reduce the health risks associated with weight gain and obesity, while minimizing evasiveness and the incidence of adverse side effects in the mammalian subjects to which they are administered.

### SUMMARY OF THE INVENTION

The present invention addresses and resolves all of the quandaries associated with contemporary weight control products. The present invention relates to compositions comprising one or more agents that stimulate mammalian lipolysis and/or modulate mammalian thermogenesis, while minimizing adverse health risks and the evasiveness associated with their administration. In one aspect of the present invention, compositions for controlling mammalian weight comprising one or more lipolytic and/or thermogenic agents are provided. Said lipolytic and/or thermogenic agents are selected from the group consisting of: theophylline, rolipram, amrinone, zardaverine, theobromine, soy lipolytics, soy isoflavones, genistein, daidzein, green tea catechins, epigallocatechin gallate, epigallocatechin, fish oil, (all-Z)-4,7,10,13,16,19-Docosahexaenoic acid, (all-Z)-5,8,11,14,17-Eicosapentaenoic acid, linoleic acid, oleate, ethyl oleate, coenzyme A, cryptotanshinone, keeman black tea and combinations thereof. In another aspect of the present invention, products comprising one or more of the compositions of the present invention are provided. In yet still another aspect of the present invention, methods of stimulating mammalian lipolysis and/or modulating mammalian thermogenesis via use of one or more compositions and/or products provided herein are disclosed. As will become apparent from the present disclosure, the combined and systematic use of the compositions, products and methods disclosed herein results in the conveyance of one or more weight control benefits, including but not limited to: localized weight loss, body sculpting, and/or anti-cellulite benefits.

### DETAILED DESCRIPTION OF THE INVENTION

Definitions and Usage of Terms

As used herein, the phrase “weight control” is intended to refer to the conveyance of one or more benefits, including but not limited to: localized weight loss, body sculpting, anti-cellulite benefits, skin firming benefits and reduction of subcutaneous fat.

As used herein, the term “lipolysis” is intended to refer to the hydrolysis of triglyceride as measured the release of glycerol and/or free fatty acid from adipocyte cultures.

As used herein, the phrase “stimulate mammalian lipolysis” is intended to refer to the ability of the present lipolytic agents to increase the rate of triglyceride hydrolysis in cultured adipocytes as measured by glycerol release or free fatty acid release from adipocyte cultures.

As used herein , the term “thermogenesis” is intended to refer to the movement of free fatty acids into the β-oxidation pathway, as measured indirectly through increases in oxygen consumption.

As used herein, the phrase “modulate mammalian thermogenesis” is intended to refer to the ability of the present thermogenic agents to stimulate an increase in oxygen consumption in isolated adipocyte cultures.

First Aspect—Compositions for Stimulating Mammalian Lipolysis & Modulating Thermogenesis

Adipocyte-Based Assay Used to Identify Lipolytic Agents

The lipolytic agents of the present invention were identified using customized adipocyte-based assays developed specifically to test the activity of various agents in the stimulation of lipolysis in mammalian adipoctyes. In general, the lipolysis assay used adherent cells. Pre-adipocytes (ex. 3T3-F442A) were seeded at a density of 1×10⁵ cells/cm²/well of a 96-well plate. After two days, when the cell reached confluence, the growth medium was replaced with differentiation medium (DMEM, 10% FBS, 5 μg/ml insulin). Half of the differentiation medium was removed and replaced with fresh differentiation medium every other day for a total of ten days, at which time differentiation progressed sufficiently such that the cells were responsive to certain lipolytic compounds. The buffer was changed to Minimal Essential Media with 4.0% BSA (lipolysis assay medium), and the cells were in condition for placement in the assay. For example, forskolin (10 μM) or norepinephrine (100 nM) were added to the differentiated 3T3-F442A adipocytes, incubated for a total of 3-6 hours in lipolysis assay medium and 100 μl of medium was collected and the amount of glycerol released into the medium was used as a measure of lipolysis.

The lipolysis assay was also developed in differentiated human adipocytes. Human adipocytes were purchased in 96-wells as 7-day differentiated cells (Zen Bio Inc.). According to manufacturer's instruction, adipocytes were incubated in the original media until day 15 for the adipocytes to mature. The lipolysis assay was performed on day 15. The lipolysis assay was also run with rat primary adipocytes. The rat adipocytes were isolated from epididymal fat pads and placed into Krebs-Ringer HEPES Buffer with 3.5% BSA, 0.55 mM D-glucose and 200 nM Adenosine. They were then minced and collagenase was added to a final concentration of 1 mg/mL type IV or type I Collagenase. This suspension was rocked for about 1 hour at 37° C. then filtered through fine nylon mesh and washed. The samples were repeatedly washed with Krebs-Ringer HEPES Buffer with 3.5% BSA, and 200 nM Adenosine. Finally, they were suspended in lipolysis assay medium at a density of about 1×10⁵ cells/mL and assayed using 1 mL of adipocyes. 50 microliters were collected for glycerol analysis at various times during the assay.

Agents for Stimulating Mammalian Lipolysis

The above-described assays were used to identify several agents that demonstrate meaningful activity in the stimulation of lipolysis in human and/or rat adipocytes. Thus, in accordance with a first aspect of the present invention, compositions for stimulating mammalian lipolysis, identified using the above-described assay, are provided. In one aspect, the compositions of the present invention may comprise two or more lipolytic agents selected from the group consisting of: soy lipolytics, theophylline, rolipram, amrinone, zardaverine, theobromine, cantharidin [CAS # 56-25-7], 2-methoxy-6-[[[4-(4-methoxyphenyl)-5-(4-nitrophenyl)-2-thiazolyl]imino]methyl]-phenol [CAS # 413573-52-1], 4-(4-methoxyphenyl)-5-(4-nitrophenyl)-2-thiazolamine [CAS # 310457-55-7] and combinations thereof. Suitable soy lipolytics for purposes of the present invention are selected from the group consisting of: soy isoflavones, genistein, daidzein and combinations thereof. The forgoing enumeration of lipolytic agents suitable for use in the context of the present invention is provided only for purposes of illustration, and is in no way intended to limit the scope of the present invention. In other aspects of the present invention, reasonable variants and derivatives of the above-listed lipolytic agents may be incorporated alone and/or in combination with one or more of the above-listed lipolytic agents.

Indeed, it has been surprisingly discovered and documented via the present disclosure, that the above-identified lipolytic agents are effective in stimulating mammalian lipolysis. By “effective,” it is meant that the above-described lipolytic agents exhibited stimulation of lipolysis in mammalian adipocytes by at least 15% as compared to forskolin, when a standard dose response cure is performed. Without wishing to be bound by theory, it is believed that the lipolytic agents of the present invention function by activating processes in the cells that increase the activity of Hormone Sensitive Lipase—thereby increasing the rate at which the triglycerides in the adipocytes are broken down.

Preferred Ratios, Levels and Combinations of Lipolytic Agents

In one aspect of the present invention, the compositions of the present invention may comprise two or more of the above-listed lipolytic agents. In another aspect of the present invention, the lipolytic agents of the present invention may be present in a ratio of from about 2:1 to about 1:1, to each other. In another aspect of the present invention, the lipolytic agents disclosed herein can be present in a ratio of from about 1.5:1 to about 1:1, to each other. In yet another aspect of the present invention, the lipolytic compounds disclosed herein may be present at a level of from about 0.0001% to about 10% by weight of the entire composition. In yet still another aspect of the present invention the lipolytic agents disclosed herein may be present at a level of from about 0.0001% to about 5% by weight of the entire composition. In another aspect of the present invention, the lipolytic agents disclosed herein may be present at a level of from about 0.001% to about 3% by weight of the entire composition. In yet another aspect of the present invention, the lipolytic agents disclosed herein may be present at a level of from about 0.1% to about 2% by weight of the entire composition.

In another aspect of the present invention, the lipolytic compositions of the present invention may comprise genistein and daidzein. In one aspect, the compositions of the present invention may comprise from about 10 mg/kg to about 30 mg/kg, more preferably from about 15 mg/kg to about 25 mg/kg, most preferably about 20 mg/kg, of each of genistein and daidzein. The preceding aspect is particularly useful in the oral administration of the present compounds, as discussed infra. In another aspect of the present invention, the lipolytic compositions of the present invention may comprise theophylline and genistein. In one aspect of the present invention, the lipolytic compositions disclosed herein may comprise from about 0.25% to about 5%, more preferably from about 0.5% to about 2%, most preferably about 1% theophylline, and from about 0.5% to about 10%, preferably from about 1% to about 5%, more preferably from about 1.5% to about 3.5%, most preferably about 2% genistein. The preceding ratios, levels and combinations are in no way intended to limit the scope of the present invention. Those skilled in the art to which the subject invention relates will appreciate that the precise level and/or ratio of lipolytic actives included in the compositions of the present invention will depend upon a variety of factors, including but not limited to: the needs and/or abilities of the formulator and/or practitioner; and the purpose for which use of the compositions and products disclosed herein is intended.

Adipocyte-Based Assay Used to Identify Thermogenic Agents

Thermogenesis was measured using an oxygen probe to monitor oxygen consumption by the adipocytes as they oxidize fatty acids. This assay was developed in primary rat adipocytes (isolated as above) using a YSI 5300 monitor equipped with oxygen-sensitive electrodes in an 8-chamber air tight apparatus. The assay was validated with norepinephrine, which is known to increase the rate of fatty acid oxidation and oxygen consumption. Since oxygen consumption can be induced by both the Kreb's cycle (glucose metabolism) and β-oxidation (fatty acid metabolism), fatty acid oxidation inhibitors were used to test whether the increase in oxygen consumption is indeed induced by fatty acid oxidation. Valproic acid inhibits the entry of fatty acid into the mitochondria and 4-pentenoic acid inhibits β-ketothiolase (the enzyme in the last step of β-oxidation). Both were found to inhibit oxygen consumption induced by norepinephrine, demonstrating that the measurement of oxygen consumption correlates with fatty acid oxidation.

Agents for Modulating Mammalian Thermogenesis

In another aspect of the present invention, agents for modulating mammalian thermogenesis, identified via use of the above-described assay, are provided. In one aspect, the thermogenic compositions of the present invention may comprise two or more thermogenic agents selected from the group consisting of: green tea catechins, epigallocatechin gallate, epigallocatechin, fish oil, (all-Z)-4,7,10,13,16,19-Docosahexaenoic acid, (all-Z)-5,8,11,14,17-Eicosapentaenoic acid, linoleic acid, oleate, ethyl oleate, coenzyme A, cryptotanshinone, keeman black tea and combinations thereof. The preceding list is in no way intended to limit the scope of the present invention. The present invention seeks to encompass reasonable variants and/or derivatives of the above- enumerated thermogenic agents, alone or in combination with one or more of the aforementioned thermogenic agents.

It has been discovered that the use of the above-listed thermogenic agents in accordance with the present invention results in the effective modulation of thermogenesis in mammals. By “effective,” it is meant that administration of the thermogenic agents of the present invention results in a 10 to 15% increase in thermogenic activity in the in vitro oxygen consumption assay. Without wishing to be bound by theory, it is believed that the thermogenic agents of the present invention function by increasing the rate at which fatty acids are transported into the mitochondria and oxidized in the cell.

Preferred Ratios, Levels and Combinations of Thermogenic Agents

In one aspect of the present invention, compositions comprising two or more of the above-enumerated thermogenic agents are provided. In one aspect of the present invention, the thermogenic agents disclosed herein may be preset in a ratio of from about 2:1 to about 1:1, to each other. In yet another aspect of the present invention, compositions comprising two or more of the thermogenic agents disclosed herein may be present in a ratio of about 1:1, to each other. In another aspect of the present invention, compositions for modulating thermogenesis in accordance with the present invention may comprise from about 0.0001% to about 10%, preferably from about 0.001% to about 5%, more preferably from about 0.01% to about 3%, most preferably from about 0.1% to about 2%, by weight of the entire thermogenic composition, of two or more thermogenic agents, as disclosed herein.

In another aspect of the present invention, the thermogenic compositions of the present invention may comprise green tea catechins. In one aspect of the present invention, the thermogenic compositions of the present invention may comprise from about 15 mg/kg to about 60 mg/kg, more preferably from about 20 mg/kg to about 50 mg/kg, most preferably about 40 mg/kg, of a green tea catechin compound. Such an embodiment is particularly suitable for the oral administration of the present compounds, as discussed infra. In yet another aspect of the present invention, the thermogenic compositions of the present invention may comprise a green tea catechin compound in combination with at least one other thermogenic agent in accordance with the present invention. The preceding ratios, levels and combinations are in no way intended to limit the scope of the present invention. Those skilled in the art to which the present invention relates will appreciate that the precise level and/or ratio of thermogenic agents included in the compositions of the present invention will depend upon a variety of factors, including but not limited to, the needs and/or abilities of the formulator and/or practitioner; and the purpose for which use of the thermogenic agents and compositions disclosed herein is intended.

Adipocyte-Based Assay Demonstrating Activity of Lipolytic & Thermogenic Agent Combinations

Freshly-islolated rat adipocytes were diluted to a concentration of 1×10⁵ cells/mL, and then 3 mL of the adipocyte suspension was placed into glass vials to be used with the YSI 2100 Oxygen Monitor. This system measures dissolved oxygen and can be used to monitor oxygen consumption in a suspension of isolated rat adipocytes. It has been used to demonstrate that the adipocyte-derived hormone, leptin, stimulates beta-oxidation in adipoctyes. Compounds are added to the adipocyte suspension. Oxygen probes are fitted into the sample chamber eliminating air from the chamber, and changes in dissolved oxygen over an hour period are measured while a stir bar gently mixes the solution. Changes in oxygen consumption in these assays are due to the beta-oxidation of free fatty acids as several inhibitors of beta-oxidation (at different points in the beta-oxidation pathway) prevent oxygen consumption by exogenous agents. The activity of compounds in the oxygen consumption assays is compared to the positive controls, coenzyme A or 200 nM norepinephrine. Following the oxygen consumption assay, medium is collected and lipolytic activity is determined by the amount of glycerol released. The general trend observed is that when lipolytic agents are mixed in vitro with agents that stimulate beta-oxidation, both activities are observed, and sometimes beta-oxidation is enhanced when the combination of lipolytic and thermogenic agents is placed together.

Compositions Comprising Lipolytic and Thermogenic Agents

In yet another aspect of the present invention, compositions comprising one or more lipolytic agents and one or more thermogenic agents, identified using the above-described assay(s), are provided. In one aspect of the present invention, the compositions disclosed herein may comprise at least one lipolytic agent and at least one thermogenic agent selected from the group consisting of: theophylline, rolipram, amrinone, zardaverine, theobromine, soy lipolytics, soy isoflavones, genistein, daidzein, green tea catechins, epigallocatechin gallate, epigallocatechin, fish oil, (all-Z)-4,7,10,13,16,19-Docosahexaenoic acid, (all-Z)-5,8,11,14,17-Eicosapentaenoic acid, linoleic acid, oleate, ethyl oleate, coenzyme A, cryptotanshinone, keeman black tea, and combinations thereof. In another aspect of the present invention, the compositions of the present invention may comprise from about 0.0001% to about 5% of a lipolytic agent and from about 0.0001% to about 5% of a thermogenic agent, by weight of the entire composition. In another aspect of the present invention, the compositions disclosed herein may comprise from about 0.001% to about 3% of a lipolytic agent and from about 0.001% to about 3% of a thermogenic agent, by weight of the entire composition. In yet another aspect of the present invention, the compositions disclosed herein may comprise from about 0.1% to about 2% of a lipolytic agent and from about 0.1% to about 2% of a thermogenic agent, by weight of the entire composition. In yet still another aspect of the present invention, the compositions disclosed herein may comprise from about 0.5% to about 2%, preferably about 1% of theophylline; from about 1% to about 3%, preferably about 2% of genistein; and from about 2% to about 10%, preferably about 5% of green tea catechins, by weight of the entire composition. The preceding composition is particularly useful in the topical administration of the claimed compounds, as discussed infra.

In yet still other aspect of the present invention, the compositions disclosed herein may comprise from about 10 mg/kg to about 30 mg/kg of genistein, 10 mg/kg to about 30 mg/kg of daidzein, and from about 30 mg/kg to about 50 mg/kg of green tea catechins. The preceding composition is particularly suitable for the oral administration of the present compositions, as discussed infra. In yet another aspect of the present invention, the compositions disclosed herein may comprise about 20 mg/kg of genistein, 20 mg/kg of daidzein and about 40 mg/kg of green tea catechins. The preceding composition is particularly useful in the oral administration of the present compositions. In another aspect of the present invention, the compositions disclosed herein may comprises from about 40 mg/kg of genistein, daidzein, and/or soy isoflavones and about 40 mg/kg of green tea catechins. In yet still another aspect of the present invention, the compositions disclosed herein may comprise from about 20 mg/kg to about 60 mg/kg, preferably from about 30 mg/kg to about 50 mg/kg, most preferably about 40 mg/kg of genistein and from about 20 mg/kg to about 60 mg/kg, preferably from about 30 mg/kg to about 50 mg/kg, most preferably about 40 mg/kg of green tea catechins. The preceding composition, too, is particularly effective in the oral administration of the present compositions.

Second Aspect: Products Comprising Lipolytic and/or Thermogenic Compositions

In another aspect of the present invention products, consumer and otherwise, comprising the lipolytic and/or thermogenic compositions of the present invention, as well as combinations of such products are provided. Indeed, the combined and systematic use of products containing the lipolytic agents and/or thermogenic agents of the present invention serves to convey several, weight control benefits to the mammalian subjects to which they are applied, including but not limited to localized weight loss, body sculpting and/or anti-cellulite benefits. The products of the present invention may take a variety of shapes and forms, depending on the specific needs and/or abilities of the practitioner of the present invention, as well as the purpose for which their employment is intended. Suitable, but non-limiting product forms, include emulsions, gels, lotions, creams, ointments, mousses, sprays, mists, sticks, powders, wraps, capsules, pills and combinations thereof

The amount of lipolytic and/or thermogenic agents incorporated into the products of the present invention will depend on the purpose for which employment of the subject product is desired. In one aspect of the present invention, the products of the present invention may comprise from about 0.001% to about 20%, preferably from about 0.001% to about 10%, more preferably from about 1% to about 5%, by weight of the entire product, of a lipolytic agent. In another aspect of the present invention, the products disclosed herein may comprise from about 0.0001% to about 20%, preferably from about 0.001% to about 10%, more preferably from about 1% to about 5%, by weight of the entire product, of a thermogenic agent. In yet another aspect of the present invention, the products disclosed herein may comprise a combination of one or more lipolytic agents and one or more thermogenic agents, as defined herein. In such an instance, the present products may comprise from about 0.001% to 20%, preferably from about 0.001% to about 10%, more preferably from about 1% to about 5% by weight of a lipolytic agent and from about 0.001% to about 20%, preferably from about 0.001% to about 10%, more preferably from about 1% to about 5%, by weight, of a thermogenic agent.

Products for Topical Administration of Lipolytic and/or Thermogenic Agents

In another aspect of the present invention, the lipolytic agents and/or thermogenic agents disclosed herein may be formulated into a product for topical application onto a mammalian subject. In one aspect of the present invention the topical formulations disclosed herein may comprise a safe and effective amount of lipolytic agents and/or thermogenic agents, as disclosed herein. By “safe and effective amount”, it is intended that an incorporated amount of a lipolytic and/or thermogenic agent be high enough to convey one or more weight control benefits, but low enough to discourage adverse side effects. Indeed, the safe and effective amount of an agent for use in the compounds and/or compositions of the present invention will vary depending on one or more of the following factors: the nature of the skin for which treatment is sought, the age and physical condition of the mammal for which administration is intended, the severity of any existing skin conditions, the intended duration of the treatment, the existence and nature of any concurrent therapy, the particular lipolytic and/or thermogenic agent for which administration is intended, the particular excipients utilized, and the needs and/or abilities of the formulator of the present compositions and products. Nevertheless, the appropriate amount of the agent to be incorporated into the present compositions may be determined by routine experimentation with animal models. Indeed, one such model includes, but certainly is not limited to, intact and aged murine models of mammalian, and particularly human, skin.

In one aspect of the present invention, the products disclosed herein take the form of a wrap or transdermal patch product, particularly suitable for wrapping and/or covering a portion of mammalian skin for which the conveyance of one or more weight control benefits is desired. Wraps and/or transdermal patches suitable for use in the context of the present invention include those marketed by The Procter and Gamble Company, Cincinnati, Ohio. In such instance, the lipolytic agents and/or thermogenic agents of the present invention are preferably embedded or impregnated into said wrap product.

In another aspect of the present invention, products adapted for topical administration of the lipolytic agents and/or thermogenic agents of the present invention, are provided. In one aspect of the present invention, the lipolytic agents and/or thermogenic agents disclosed herein are incorporated into a topical skin care product, including but not limited to, lotions, creams, gels and ointments, particularly those marketed by The Procter and Gamble Company of Cincinnati, Ohio. In one aspect, the topical products disclosed herein may comprise from about 0.0001% to about 10%, preferably from about 0.0001% to about 5%, more preferably from about 0.001% to about 3%, most preferably from about 0.1% to about 2%, by weight of the entire topical product, two or more lipolytic agents, as defined herein. In yet still another aspect of the present invention, the topical products disclosed herein may comprise from about 0.0001% to about 10%, preferably from about 0.001% to about 5%, more preferably from about 0.01% to about 3%, most preferably from about 0.1% to about 2%, by weigh to the entire product, of two or more thermogenic agents, as defined herein. In yet still another aspect of the present invention, the topical products disclosed herein may comprise from about 0.0001% to about 5%, preferably from about 0.001% to about 3%, more preferably from about 0.1% to about 2% by weight of the entire product of a lipolytic agent; and from about 0.0001% to about 5%, preferably from about 0.001% to about 3%, more preferably from about 0.1% to about 2%, by weight of the entire product of a thermogenic agent. In yet another aspect of the present invention, the topical products disclosed herein may comprise from about 0.5% to about 2%, preferably about 1% of theophylline; from about 1% to about 3%, preferably about 2% of genistein; and from about 2% to about 10%, preferably about 5% of green tea catechins, by weight of the entire topical product.

In another aspect of the present invention, the topical products of the present invention may comprise certain adjunct ingredients. Said adjuncts include, but certainly are not limited to: antimicrobial and antifungal actives, surfactants, desquamation actives, anti-acne actives, anti-wrinkle actives, anti-atrophy actives, anti-oxidants, radical scavengers, chelators, flavonoids, anti-inflammatory agents, anti-cellulite agents, topical anesthetics, tanning actives, sunscreen actives, conditioning agents, thickening agents, detackifying agents, odor control agents, skin sensates, antiperspirants and mixtures thereof. Indeed, a complete description and examples of each of the aforementioned adjunct ingredients is set forth in U.S. Pat. No. 6,294,186, assigned to The Procter and Gamble Company, Cincinnati, Ohio and incorporated herein by reference. Indeed, the precise form of suitable skin care products for use in combination with the lipolytic agents and/or thermogenic agents of the present invention will depend on the needs and/or abilities of the formulator of the present compositions and products.

In one aspect of the present invention, topical compositions comprising the lipolytic agents and/or thermogenic agents disclosed herein may contain a dermatologically acceptable carrier. The phrase “dermatologically-acceptable carrier”, as used herein, is intended to mean that the carrier is suitable for topical application to the keratinous tissue, has good aesthetic properties, is compatible with the actives of the present invention and any other components, and will not cause any untoward safety or toxicity concerns. A safe and effective amount of carrier is from about 50% to about 99.99%, preferably from about 80% to about 99.9%, more preferably from about 90% to about 98%, and even more preferably from about 90% to about 95% of the composition. The carrier can be in a wide variety of forms. For example, emulsion carriers, including, but not limited to, oil-in-water, water-in-oil, water-in-oil-in-water, and oil-in-water-in-silicone emulsions, are useful herein. Emulsions according to the present invention generally contain a solution as described above and a lipid or oil. Lipids and oils may be derived from animals, plants, or petroleum and may be natural or synthetic (i.e., man-made).

Preferred emulsions further contain a humectant, such as glycerin. Emulsions may further contain from about 0.01% to about 10%, more preferably from about 0.1% to about 5%, of an emulsifier, based on the weight of the carrier. Emulsifiers may be nonionic, anionic or cationic. Suitable emulsifiers are disclosed in, for example, U.S. Pat. No. 3,755,560, issued Aug. 28, 1973, Dickert et al.; U.S. Pat. No. 4,421,769, issued Dec. 20, 1983, Dixon et al.; and McCutcheon's Detergents and Emulsifiers, North American Edition, pages 317-324 (1986). The emulsion may also contain an anti- foaming agent to minimize foaming upon application to the keratinous tissue. Anti-foaming agents include high molecular weight silicones and other materials well known in the art for such use.

The products of the present invention may optionally contain one or more additional skin care actives or combination of skin care actives to enhance topical administration. The skin care active may be included as a substantially pure material, or as an extract obtained by suitable physical and/or chemical isolation from natural sources. In one aspect, where the composition is to be in contact with human keratinous tissue, the additional skin care active(s) should be suitable for application to keratinous tissue, that is, when incorporated into the composition they are suitable for use in contact with human keratinous tissue without undue toxicity, incompatibility, instability, allergic response, and the like within the scope of sound medical judgment. The CTFA Cosmetic Ingredient Handbook, Second Edition (1992) describes a wide variety of cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropynyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, thickeners, and vitamins and derivatives thereof.

In yet still another aspect of the present invention, topical delivery of the present lipolytic agents is enhanced via use of 1-methyl-2-pyrrolidinone. In yet another aspect of the present invention, topical delivery of the lipolytic agents disclosed herein is enhanced via use of 1-methyl-2-pyrrolidinone, transcutol and lauryl alcohol, as described in the formulation chart depicted in Example 1 below. In another aspect of the present invention, the topical delivery of genistein is enhanced via use of 1-methyl-2-pyrrolidinone. In yet still another aspect of the present invention, the topical delivery of genistein is enhanced via use of 1-methyl-2-pyrrolidinone, transcutol and lauryl alcohol, as described in the formulation chart set forth in Example 1. In yet another aspect of the present invention, the topical delivery of the present lipolytic agents, and particularly genistein, is enhanced via use of 1-methyl-2-pyrrolidinone in combination with other conventional, lotion-based formulations. Particularly preferred lotion-based formulations include those marketed by The Procter and Gamble Company of Cincinnati, Ohio.

Products for Oral Administration of Lipolytic and/or Thermogenic Agents

The lipolytic agents and/or thermogenic agents of the present invention may be administered systemically, e.g., orally and/or parenterally, including subcutaneous or intravenous injection, and/or intranasally, but especially transdermally. In one aspect of the present invention, the lipolytic agents and/or thermogenic agents disclosed herein are conveyed to the intended mammalian recipient in a unit dosage form. The precise amount of the present compounds incorporated into a unit dosage form will depend upon one or more factors disclosed hereinbefore, and particularly the needs and/or abilities of the formulator of the present compositions and the nature of the mammal for which treatment is desired. In yet another aspect of the present invention, the dose forms for use with the present compounds and products include nasal, transdermal, rectal, sublingual, oral and combinations thereof. In another aspect of the present invention, one or more carriers suitable for use in the present invention may be employed to achieve delivery of the present agents and/or compositions, and particularly for injection or surgical implants. Said carriers include, but certainly are not limited to: hydrogels, controlled- or sustained release devises, polylactic acid, collagen matrices, and combinations thereof.

In one of the present invention, the compositions and products disclosed herein are administered orally. In one aspect of the present invention, the lipolytic agent-comprising compositions disclosed herein may be incorporated into a product and administered orally. In this aspect of the present invention, said product may comprise from about 10 mg/kg to about 30 mg/kg, more preferably from about 15 mg/kg to about 25 mg/kg, most preferably about 20 mg/kg, of each of genistein and daidzein. In another aspect of the present invention, the thermogenic agent-comprising compositions of the present invention may be incorporated into a product and administered orally. In this aspect of the present invention, said product may comprise from about 5 mg/kg to about 60 mg/kg, more preferably from about 20 mg/kg to about 50 mg/kg, most preferably from about 40 mg/kg, of green tea catechins. In yet still another aspect of the present invention compositions comprising both the lipolytic and thermogenic agents disclosed herein may be incorporated into a product for oral administration to a mammalian subject. In this aspect of the present invention, said compositions may comprise from about 10 mg/kg to about 30 mg/kg of daidzein, and from about 30 mg/kg to about 50 mg/kg of green tea catechins. In yet another aspect of the present invention, the products disclosed herein may comprise from about 20 mg/kg of genistein, 20 mg/kg of daidzein and about 40 mg/kg of green tea catechins. In yet still another aspect of the present invention, the products disclosed herein may comprise from about 20 mg/kg to about 60 mg/kg, preferably from about 30 mg/kg to about 50 mg/kg, most preferably about 40 mg/kg, of genistein; and from about 20 mg/kg to about 60 mg/kg, preferably from about 30 mg/kg to about 50 mg/kg, most preferably about 40 mg/kg, of green tea catechins.

Oral forms suitable for administration of the present compounds and products include, but are not limited to: liposomes, lipid emulsions, proteinaceous cages, other excipients and combinations thereof. Use of the term “excipients” herein is intended to encompass any physiologically inert, pharmacologically inactive material known to those of ordinary skill in the art. Suitable excipients for use in the present invention are compatible with the physical and chemical characteristics of the particular ingredient for which employment is sought, as well as the mammalian subject for which administration of the present compositions and/or products is intended. In one aspect of the present invention, suitable excipients for use herein include, but are not limited to, polymers, resins, plasticizers, fillers, lubricants, binders, disintegrants, solvents, co-solvents, buffer systems, surfactants, preservatives, sweetening agents, flavoring agents, fragrance agents pharmaceutical grade dyes, pigments and combinations thereof.

When the use of a flavoring agent excipient in the compositions of the present invention is desired, suitable such agents may be selected from those described in Remington's Pharmaceutical Sciences, 18th Edition, Mack Publishing Company, 1990, pp. 1288-1300, incorporated by reference herein. Dyes, or pigments suitable for use in the present invention include, but are not limited to, those described in Handbook of Pharmaceutical Excipients, Second Edition pp. 126-134, 1994 by the American Pharmaceutical Association & the Pharmaceutical Press, incorporated by reference herein.

Suitable solvents and co-solvents for use in the present invention include, but are not limited to, water, ethanol, glycerin, propylene glycol, polyethylene glycol and combinations thereof. Suitable buffer systems for use as excepients herein include, but are not limited to potassium acetate, boric carbonic, phosphoric, succinic, malic, tartaric, citric, acetic, benzoic, lactic, glyceric, gluconic, glutaric, glutamic and combinations thereof. In one aspect of the present invention suitable buffer systems for use herein are phosphoric, tartaric, citric, and potassium acetate.

Suitable surfactants for use as excepients in the present invention include, but are not limited to, polyoxyethylene sorbitan fatty acid esters, polyoxyethylene monoalkyl ethers, sucrose monoesters and lanolin esters, ethers and mixtures thereof. Moreover, suitable preservatives for use as excepients of the present invention include, but are not limited to, phenol, alkyl esters of parahydroxybenzoic acid, benzoic acid and the salts thereof, boric acid and the thereof, sorbic acid and the salts thereof, chlorobutanol, benzyl alcohol, thimerosol, phenylmercuric acetate and nitrate, nitromersol, benzalkonium chloride, cetylpyridinium chloride, methyl paraben, and propyl paraben. Particularly preferred are the salts of benzoic acid, cetylpyridinium chloride, methyl paraben, propyl paraben and combinations thereof.

Suitable sweeteners for use with the lipolytic agents and/or thermogenic agents disclosed herein include, but are not limited to, sucrose, glucose, saccharin, aspartame and combinations thereof. In another aspect of the present invention, sucrose, saccharin and combinations thereof are particularly preferred sweeteners for use with the present compounds. Suitable binders for use in conjunction with the present compounds include, but are not limited to methylcellulose, sodium carboxymethylcellulose, hydroxypropylmethylcellulose, carbomer, prodione, acacia, guar gum, xanthan gum, tragcanth and combinations thereof. In yet another aspect of the present invention, particularly preferred binders for use herein include, but are not limited to, methylcellulose, carbomer, xanthan gum, guar gum, povidone, sodium carboxymethylcellulose and combinations thereof.

Suitable fillers for use with the lipolytic agents and/or thermogenic agents disclosed herein include, but are not limited to lactose, sucrose, maltodextrin, mannitol, starch, microcrystalline cellulose and combinations thereof. Suitable plasticizers for use with the present compounds include, but are not limited to polyethylene glycol, propylene glycol, dibutylphthalate, and castor oil, acetylated monoglycerides, triactin and combinations thereof. In another aspect of the present invention, suitable lubricants for use herein include, but are not limited to, magnesium stearate, stearic acid, talc and combinations thereof. Indeed, suitable disintegrants for use with the compounds of the present invention include, but are not limited to, crospovidone, sodium carboxymethyl starch, sodium starch glycollate, sodium carboxymethyl cellulose, alginic acid, clays, ion exchange resins and combinations thereof. Suitable polymers for use as excepients of the present invention, include but are not limited to, hydroxypropylmethylcellulose (HPMC), hydroxypropylcellulose (HPC), carboxymethylcellulose, acrylic resins such as Eudragit® RL30D, manufactured by Rohm Pharma GmbH Weiderstadt, West Germany, methylcellulose, ethylcellulose, polyvinylpyrrolidone, commercially available film-coating preparations such as Dri-Klear, manufactured by Crompton & Knowles Corp., Mahwah, N.J., Opadry manufactured by Colorcon, West Point, Pa. and combinations thereof. It should be reiterated and underscored that the precise ingredients and suitable excepients for use with the compounds of the present invention will depend on several factors, and particularly the needs and/or abilities of the formulator and the nature of the mammal for which treatment with the present compounds is desired. Nevertheless, the above discussion is intended only to serve as a guide to a person of ordinary skill in the art. Certainly, compounds analogous or similar to those listed above will also be suitable for employment with the compounds of the present invention.

Articles of Manufacture & Kits

Moreover, articles of manufacture comprising the lipolytic agents and/or thermogenic agents of the present invention and/or one or more of the aforementioned products, are intended for personal care and skin care applications. The article of manufacture of the present invention encompasses one or more products as described hereinbefore that may be packaged in a container or dispenser with a set of instructions for the consumer. The article of manufacture of the present invention typically comprises (a) container or dispenser, (b) product and (c) set of instructions to administer said product to an appropriate substrate to convey one or more weight control benefits. Containers and/or dispensers suitable for the article of manufacture of the present invention include, but are not limited to: PET bottles and tubs, flow-wrap pouches, foaming dispensers, spray dispensers and combinations thereof. To reiterate, the article of manufacture of the present invention further comprises a set of instructions in association with the container. By “in association with,” it is meant that the instructions are either directly printed on the container or dispenser itself or presented in a different fashion including, but not limited to: a brochure, print advertisement, electronic advertisement and/or verbal communication, so as to communicate the set of the instructions to a consumer of the article of manufacture.

The set of instructions typically comprise the instructions relating to the use of the product to administer the lipolytic agents and/or thermogenic agents of the present invention to a mammalian subject in need of treatment. The set of instructions may further comprise the instruction to allow the present compounds to remain on the treated substrate, without rinsing or otherwise removing the compounds from the treated substrate. Nevertheless, the precise instructions included with the article of manufacture of the present invention will depend on the specific composition and/or product for which the inclusion of instructions is desired and the substrate onto which application of the product is intended. In another aspect of the present invention, the instructions included in the present articles of manufacture coincide with the methods set forth in the “Methods of Using the Present Compositions and Products” section of the present disclosure.

Third Aspect—Methods of Using the Present Compounds and Products

In yet another aspect, methods of using the compositions and/or products of the present invention are provided. The compositions and products of the present invention are useful conveying one or more weight control benefits to the mammalian subject to which they are administered including but not limited to: localized weight loss, body sculpting benefits and/or anti-cellulite benefits. When practiced in accordance with the present invention, the compositions and products disclosed herein serve to stimulate mammalian lipolysis and/or modulate mammalian thermogenesis. In one aspect of the present invention, the compounds and/or products disclosed herein are directly applied to a portion of mammalian skin for which treatment is desired. In this respect, treatment may, for example, include localized weight loss and/or anti-cellulite benefits. In another aspect of the present invention, the compositions and/or products disclosed herein are applied transdermally to the mammalian skin for which treatment is sought. In this respect, a wrap comprising the compositions and/or products of the present invention, for example, may be employed. The exact amount of the compositions and/or nature of products will depend upon the needs and abilities of the formulator and practitioner of the present methods. In another aspect of the present invention, the compounds of the present invention are administered to a mammal in need of treatment at least once per day. Once applied, the compositions are rubbed on the treated surfaces for a period of time to ensure coverage. In another aspect of the present invention, transdermal dosages are designed and intended to attain minimal serum or plasma levels, based upon techniques known to those skilled in the art of pharmacokinetics and formulations. The compositions and products of the present invention are suitable for a variety of uses. Indeed, suitable uses of the present compositions include, but certainly are not limited to, the conveyance of one or more weight control benefits as defined herein, the stimulation of mammalian lipolysis and/or the modulation of mammalian thermogenesis.

In one aspect of the present invention, a method for stimulating mammalian lipolysis is provided, said method comprising the step of administering a lipolytic agent-comprising composition and/or product in accordance with the present invention to a mammal in need of treatment. In another aspect of the present invention, the composition and/or product may be removed following its administration. In another aspect of the present invention, a method for modulating thermogenesis in a mammal is provided, said method comprising the step of administering a thermogenic-comprising composition and/or product in accordance with the present invention, to a mammal in need of treatment. In another aspect of the present invention, the thermogenic agent-comprising composition and/or product may be removed following its administration. In yet still another aspect of the present invention, a method for stimulating lipolysis and/or modulating thermogenesis in a mammal is provided. Said method comprises the step of administering a lipolytic-agent and/or thermogenic agent-comprising composition and/or product to a mammal in need of treatment. In another aspect of the present invention, the subject composition and/or product is removed following its administration.

### PREPARATIVE EXAMPLES

### Example 1

### Formulation of Lipolytic and Thermogenic Agent-Comprising Topical Product

A product comprising a lipolytic and thermogenic agent, as defined by the present disclosure, is formulated for topical administration to a mammalian subject in accordance with the following.

| | | | % COMP | |
|---|---|---|---|---|
| | | | As | Chem. |
| TRADE NAME | CTFA NAME | CAS # | Added | Content |
| Purified Water | Water | N/A | 55.505 | 55.5175 |
| Sodium Hydroxide, | Sodium Hydroxide, | 1310-73-2 | 0.025 | 0.0125 |
| 50% Solution | 50% Solution | | | |
| Glycerin | Glycerin | 56-81-5 | 6.900 | 6.9000 |
| Titanium Dioxide | Titanium Dioxide | 13463-67-7 | 0.600 | 0.6000 |
| NMP | 1-Methyl-2-Pyrrolidinone | 872-50-4 | 5.000 | 5.0000 |
| Transcutol | Diethylene Glycol | 111-90-0 | 11.000 | 11.0000 |
| | Monoethyl Ether | | | |
| Lauryl Alcohol | Lauryl Alcohol | 67762-41-8 | 2.000 | 2.0000 |
| Genistein | Genistein | | 2.000 | 2.0000 |
| Green Tea Extract | Green Tea Extract, | | 5.000 | 5.0000 |
| | 70% EGCG-MSP ™ | | | |
| Disodium EDTA | Disodium EDTA | 139-33-3 | 0.100 | 0.1000 |
| Theophylline | thoephylline | | 1.000 | 1.0000 |
| Permethyl 101A | Isohexadecane | 4390-04-9 | 3.000 | 3.0000 |
| SEFA Cottonate | Sucrose esters of | 93571-82-5 | 0.670 | 0.6700 |
| | fatty acids cottonate | | | |
| Nipigin A | Ethylparaben | 120-47-8 | 0.200 | 0.2000 |
| Montanov 68 | Glucoside wax | 54549-27-8 | 0.500 | 0.5000 |
| Cetyl Alcohol CO-1695 | Cetyl Alcohol | 36653-82-4 | 0.320 | 0.3200 |
| Adol 62 | Stearyl Alcohol | 112-92-5 | 0.480 | 0.4800 |
| Behenyl Alcohol | Behenyl Alcohol | 661-19-8 | 0.400 | 0.4000 |
| Emersol 132 | Stearic Acid | 57-11-4 | 0.100 | 0.1000 |
| Myrj 59 | PEG-100 Stearate | 9004-99-3 | 0.100 | 0.1000 |
| Nipasol M | Propylparaben | 94-13-3 | 0.100 | 0.1000 |
| Sepigel 305 | Polyacrylamide (and) | 9003-05-8, | 2.500 | 2.5000 |
| | C13-14 Isoparaffin (and) | 3055-97-8 | | |
| | Laureth-7 | | | |
| Benzyl Alcohol | Benzyl Alcohol | 100-51-6 | 0.500 | 0.5000 |
| Dow Corning 1503 | Dimethicone (and) | 9006-65-9, | 2.000 | 2.0000 |
| | Dimethiconol | 31692-79-2 | | |

Sodium Hydroxide Premix. In an appropriate beaker, the following ingredients are added and mixed until the resulting mixture is uniform: 3.000% w/w (15.0000 grams) DRO Water, 0.025% w/w (0.1250 grams) Sodium Hydroxide, 50% solution.
TiO2 Premix. In an appropriate beaker, the following ingredients are added and mixed until the resulting mixture is uniform: 5.000% w/w (25.0000 grams) DRO Water; 6.900% w/w (34.5000 grams) Glycerin; 0.600% w/w (3.0000 grams) Titanium Dioxide. In an appropriate beaker the following ingredients are mixed and heated until dissolved: 5.000% w/w (25.0000 grams) nMP; 11.000% w/w (55.0000 grams) Transcutol; 2.000% w/w (10.0000 grams) Lauryl Alcohol; and 2.000% w/w (10.0000 grams) Genistein. 5.000%w/w (25.0000 grams) Green Tea Extract is slowly added to this solution while maintaining heat and milling with Ultra-Turrax T-25 at low speed.
Water Phase. In an appropriate beaker 47.505% w/w (237.5250 grams) DRO Water is added. While mixing with a pitched-blade impeller at 250-350 rpm, the following other ingredients are gradually added: 0.100% w/w (0.5000 grams) Disodium EDTA; and 1.000% w/w (5.0000 grams) Theophylline. This solution is heated to 75° C. and maintained.
Oil Phase. In an appropriate beaker, the following ingredients are added: 3.000% w/w (15.0000 grams) Permethyl 101A; 0.670% w/w (3.3500 grams) Sefa Cottonate; 0.200% w/w (1.0000 grams) Ethylparaben; 0.500% w/w (2.5000 grams) Montanov 68; 0.320% w/w (1.6000 grams) Cetyl Alcohol; 0.480% w/w (2.4000 grams) Stearyl Alcohol; 0.400% w/w (2.0000 grams) Behenyl Alcohol; 0.100% w/w (0.5000 grams) Stearic Acid; 0.100% w/w (0.5000 grams) Myrj 59; and 0.100% w/w (0.5000 grams) Propylparaben. The resulting mixture is heated to 75° C. and maintained. At temperature, the Oil Phase is added to the Water Phase.
Combined Phases. The Oil Phase and Water Phase combination is milled with Ultra-Turrax T-25 at 9500-13500 rpm for 3 minutes. The mixture is then further mixed with a paddle mixer at ˜100 rpm. The batch is then cooled and mixed until it reaches a temperature of 60° C. At 60° C., 2.500% w/w (12.5000 grams) Sepigel 305 is added to the batch.

### Example 2

### Formulation of Lipolytic and Thermogenic Agent-Comprising Oral Product

A product comprising a lipolytic and thermogenic agent, as defined by the present disclosure, is formulated for oral administration to a mammalian subject as follows. Green tea extract and genistein are dry blended in proportions that allow packing of 175 mg (150 mg of green tea extract and 25 mg of genistein) into #2 opaque gelatin capsules.

### Example 3

### Administration of Lipolytic and/or Thermogenic Topical Product

The product formulated in accordance with Example 1 is topically administered to one or more portions of mammalian skin for which one or more weight control benefits are desired. The composition is allowed to remain on one or more portions of the mammalian skin until the composition is removed via normal living and/or bathing habits. The composition is applied to each desired portion of skin twice a day. Following a period of between four to twelve weeks of administration, material weight control benefits are observed.

### Example 4

### Administration of Lipolytic and/or Thermogenic Oral Product

The product as formulated in accordance with above-listed Example 2 is orally administered to a mammal for which the conveyance of one or more weight control benefits is desired. The product is administered to the mammalian subject twice a day, once in the morning and once in the evening, with meals, for a total 50 mg of genistein and 150 mg of green tea extract per day. Following a period of four to twelve weeks, material weight control benefits are observed.

All documents cited are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition for stimulating lipolysis and/or modulating thermogenesis in a mammal, said composition consisting essentially of two compounds selected from the group consisting of: theophylline, rolipram, amrinone, zardaverine, theobromine, soy lipolytics, soy isoflavones, genistein, daidzein, green tea catechins, epigallocatechin gallate, epigallocatechin, fish oil, (all- Z)-4,7,10,13,16,19-Docosahexaenoic acid, (all-Z)-5,8,11,14,17-Eicosapentaenoic acid, linoleic acid, oleate, ethyl oleate, coenzyme A, cryptotanshinone, keeman black tea, cantharidin [CAS # 56-25-7], 2-methoxy-6- [ [ [4-(4-methoxyphenyl)-5-(4-nitrophenyl)-2-thiazolyl]imino]methyl]-phenol [CAS # 413573-52-1], 4-(4-methoxyphenyl)-5-(4-nitrophenyl)-2-thiazolamine [CAS # 310457-55-7] and combinations thereof.

2. The composition according to claim 1, wherein said composition comprises at least one lipolytic agent and at least one thermogenic agent.

3. The composition according to claim 2, wherein said lipolytic agent and said thermogenic agent are present in a ratio of from about 2:1 to about 1:2.

4. The composition according to claim 2, wherein said lipolytic agent and said thermogenic agent are present in a ratio of about 1:1.

5. The composition according to claim 2, wherein said lipolytic agent is present at a level of from about 0.0001% to about 5% by weight of the entire composition.

6. The composition according to claim 2, wherein said lipolytic agent is present at a level of from about 0.001% to about 3% by weight of the entire composition.

7. The composition according to claim 2, wherein said lipolytic agent is present at a level of from about 0.1% to about 2% by weight of the entire composition.

8. The composition according to claim 2, wherein said thermogenic agent is present at a level of from about 0.0001% to about 5% by weight of the entire composition.

9. The composition according to claim 2, wherein said thermogenic agent is present at a level of from about 0.001% to about 3% by weight of the entire composition.

10. The composition according to claim 2, wherein said thermogenic agent is present at a level of from about 0.1% to about 2% by weight of the entire composition.

11. The composition according to claim 1, wherein said composition further comprises a dermatologically-acceptable carrier adapted to convey said compounds to a subcutaneous portion of mammal skin onto which said compounds are applied.

12. The composition according to claim 1, wherein said composition further comprises a skin care active.

13. The composition according to claim 1, wherein said product comprises a form adapted for a type of administration selected from the group consisting of: topical, nasal, transdermal, sublingual, oral and combinations thereof.

14. The composition according to claim 13, wherein said composition takes the form adapted for topical administration and further comprises 1-methyl-2-pyrrolidinone.

15. The composition according to claim 14, wherein said composition further comprises:
a. about 5% 1-methyl-2-pyrrolidinone by weight of the entire composition;
b. about 11% diethylene glycol monoethyl ether by weight of the entire composition; and
c. about 2% lauryl alcohol by weight of the entire composition.

16. The composition according to claim 1, wherein said product is adapted for topical administration and comprises a form selected from the group consisting of: lotions, creams, gels, ointments and combinations thereof.

17. The composition according to claim 1, wherein said product is adapted for topical administration and is provided in the form of a wrap suitable for placement onto a portion of mammalian skin.

18. The composition according to claim 1, wherein said wrap comprises a material selected from the group consisting of: woven materials, non-woven materials, film-based materials, elastics and combinations thereof.
